(19) United States
(12) Patent Application Publication
(10) Pub. No.: US 2009/0064351 A1
(43) Pub. Date: Mar. 5, 2009

(54) TRANSGENIC BIRD PRODUCING ERYTHROPOIETIN AND METHOD OF CONSTRUCTING THE SAME

(75) Inventors: Shinji Iijima, Aichi (JP); Masamichi Kamihira, Fukuoka (JP); Kenichi Nishijima, Aichi (JP)

Correspondence Address: SUGHRUE MION, PLLC, 2100 PENNSYLVANIA AVENUE, N.W., SUITE 800, WASHINGTON, DC, 20037, US

(73) Assignee: Kaneka Corporation, Osaka-shi, Osaka (JP)

(21) Appl. No.: 11/910,327

(22) PCT Filed: Feb. 24, 2006
(86) PCT No.: PCT/JP2006/303398

§ 371 Date: Dec. 17, 2007

(30) Foreign Application Priority Data
Mar. 30, 2005 (JP) 2005-097792

(51) Int. Cl. C12P 21/00; A01K 67/027; C12N 5/06; C12N 15/63

(52) U.S. Cl. 800/5; 800/19; 800/4; 435/349; 800/23

(57) ABSTRACT

The present invention has its object to provide a transgenic bird producing erythropoietin at high concentration levels as well as a method for constructing the same. The present invention provides a G0 transgenic chimera bird as obtained by incubating a fertilized avian egg, infecting the early embryo formed after egg laying, except for the blastoderm stage immediately following egg laying, with a replication-deficient retroviral vector containing a foreign erythropoietin gene and allowing the embryo to hatch.

### TECHNICAL FIELD

The present invention relates to a method for constructing a genetically engineered (transgenic) bird allowing high-level expression of a useful physiologically active protein utilizable as a drug or the like in eggs laid thereby, for instance, by manipulating the genome of the corresponding bird carrying the gene for the protein, in a functioning condition, in a chromosome thereof for the production of the protein at low cost, and to a transgenic bird obtained by that method.

### BACKGROUND ART

In recent years, a number of protein drugs have come into use. This is because the gene recombination technology for introducing the genes for desired proteins into microorganisms or cultured cells has been developed and applied and it has become possible to commercially produce the proteins by cultivating such genetically modified organisms. Thus, erythropoietin, one of the glycoproteins, is produced using, as hosts, animal cell-derived cultured cells having a glycosylating function. However, this method of production has a drawback in that the cost of production is high.

It is the utilization of transgenic animals that has so far been expected as the technique for producing those proteins producible only in animal-derived cultured cells at low cost. The development of transgenic mammals went ahead. However, it cannot always be said that the industrial utilization of these animals is advantageous, since a long period of time is required for their maturation and a large breeding space is required. On the contrary, domestic fowls, typically chickens and quails, are advantageous in that the time required for their maturation is short and a limited space is sufficient for breeding them; therefore the utilization of transgenic birds derived therefrom in producing a useful protein was greatly expected.

Patent Document 1 and 2 disclose human erythropoietin-producing transgenic birds constructed using a replication-deficient avian leukosis virus (ALV) vector. However, the yield is as low as 70 ng/ml.

The erythropoietin-producing transgenic birds disclosed in Patent Documents 1 and 2 have a problem in that the yield of erythropoietin is very low.

Patent Document 1: United States Patent Application Publication 2004/0019922

Patent Document 2: United States Patent Application Publication 2004/0019923

### SUMMARY OF THE INVENTION

Accordingly, it is an object of the present invention to provide a transgenic bird producing erythropoietin at high concentration levels as well as a method for constructing the same.

The present inventors made intensive investigations and, as a result, found that a transgenic bird producing erythropoietin at high concentration levels can be obtained by the method which comprises incubating a fertilized avian egg, infecting the early embryo formed after egg laying, except for the blastoderm stage immediately following egg laying, with a replication-deficient retroviral vector containing a foreign erythropoietin gene and allowing the embryo to hatch. Such finding has now led to completion of the present invention.

### DETAILED DESCRIPTION OF THE INVENTION

The bird to be used in the practice of the invention is not particularly restricted but includes those domestic fowls and pet birds which are reared for meat and egg production, for example chickens, turkeys, ducks, ostriches and quails. Among them, chickens and quails are preferred since they are readily available and are prolific layers.

The foreign erythropoietin gene to be used in the practice of the invention is not particularly restricted but preferably is a mammal-derived one, more specifically a human-derived one or one derived from a pet animal such as a dog, for instance.

For expression thereof in avian cells, the foreign erythropoietin gene to be used in the practice of the invention is preferably connected to an appropriate promoter on the downstream side thereof.

As the promoter, there may be mentioned non-tissue-specific promoters always active in all avian somatic cells or tissue-specific promoters active only in specific avian tissue(s)/cells.

The use of such a non-tissue-specific promoter as mentioned above is advantageous since erythropoietin is expressed in blood as well and therefore can be detected already at the nestling stage.

The non-tissue-specific promoter is not particularly restricted but may be, for example, the chicken β-actin promoter. Mention may also be made of such virus-derived promoters as the simian virus 40 (SV40) promoter, cytomegalovirus (CMV) promoter and Rous sarcoma virus (RSV) promoter.

The use of such a tissue-specific promoter as mentioned above is advantageous in that the possibility of the foreign erythropoietin exerting an inhibitory action on the development of the bird and rendering high-level expression thereof difficult can be eliminated.

The tissue-specific promoter is not particularly restricted but includes, among others, the ovalbumin promoter (in particular chicken ovalbumin promoter), lysozyme promoter, ovotransferrin promoter and ovomucoid promoter. The use of such an albumen-specific promoter is preferred since erythropoietin can be expressed at high levels only in albumen.

The foreign erythropoietin gene to be used in the practice of the invention may have a posttranscriptional regulatory element added thereto. A posttranscriptional regulatory element occurring in the mRNA formed upon gene transcription is known to contribute to stable maintenance of the mRNA.

The posttranscriptional regulatory element is not particularly restricted but may be, for example, WPRE (woodchuck hepatitis virus-derived posttranscriptional regulatory element, U.S. Pat. No. 6,136,597).

For the secretion of erythropoietin, the erythropoietin gene is preferably provided with a secretory signal sequence, which is not limited to the autologous sequence.

The foreign erythropoietin gene to be used in the practice of the invention may have a marker gene added thereto.

The marker gene is not particularly restricted but includes, among others, the gene coding for a fluorescent protein such as the green fluorescent protein (GFP), the β-galactosidase gene and the neomycin resistance (Neo^{r}) gene.

The retroviral vector to be used in the practice of the invention is not particularly restricted but may be, for example, one derived from a retrovirus or lentivirus the host of which is a mouse. In particular, one derived from the Moloney murine leukemia virus (MoMLV), which is utilized in human gene therapy and is highly safe, is preferred. From the viewpoint of exclusion of the possibility of retroviral vector replication, any virus the host of which is not a bird, for example the murine stem cell virus (MSCV), is preferred.

From a safety aspect, the retroviral vector to be used in the practice of the invention is one deficient in autonomously replicating ability as a result of deletion of one or all of the gag, pol and env genes necessary for the replication of virus particles.

For efficient infection of avian cells with the viral vector, the retroviral vector to be used in the practice of the invention preferably is, but is not limited to, a pseudotype one resulting from artificial substitution of the envelope protein env with the bovine vesicular stomatitis virus VSV-G.

A preferred mode of constructing the retroviral vector to be used in the practice of the invention is now described.

Such an element or elements favorable for gene expression as a promoter and/or a marker gene is added to the foreign erythropoietin gene; the whole has a structure sandwiched between the LTRs (5′LTR and 3′LTR) of the virus mentioned above. The LTR sequences are recognized as the both ends of the gene to be inserted into a host chromosome.

Further, this gene structure comprises a virus packaging signal sequence derived from the virus mentioned above. The virus packaging signal sequence functions as a signal for packaging of virus particles.

The LTRs have promoter and terminator activities as well. For completely eliminating the possibility of retroviral vector replication, however, it is desirable that the LTRs show no promoter activity in avian cells. Preferred from this viewpoint are LTRs derived from a virus the host of which is other than a bird, for example is a mouse.

Such gene structure is provided by a single construct. The details are as described in an earlier disclosure made by the present inventors (Japanese Kokai Publication 2002-176880).

Now, a preferred mode of preparation of the replication-deficient pseudotype retroviral vector to be used in the practice of the invention is described.

The erythropoietin gene and VSV-G gene are cointroduced into cells of the so-called packaging cell line retaining the gag and pol genes among the gag, pol and env genes necessary for the replication of virus particles, and the culture supernatant is used as a virus-containing solution. Or, desirably, the VSV-G gene is introduced into stable packaging cells containing a large number of copies of the erythropoietin gene integrated into the chromosome as obtained by infecting packaging cells with the thus-prepared viral vector, and the culture supernatant is used as a virus-containing solution. The culture supernatant is desirably one prepared 2 to 3 days after gene transfer and, if necessary, it is concentrated for use as a virus-containing solution. The procedure for obtaining a virus-containing solution is not limited to any of those mentioned above.

The replication-deficient retroviral vector titer in the above virus-containing solution is preferably not lower than 1×10⁸ cfu/ml but not higher than 1×10¹⁰ cfu/ml, more preferably not lower than 1×10⁹ cfu/ml but not higher than 1×10¹⁰ cfu/ml. A virus-containing solution with such a high titer level can be obtained with ease by ultracentrifugation concentration of the virus-containing solution obtained as mentioned above utilizing the effect of the posttranscriptional regulatory element mentioned above.

The titer of the virus-containing solution is defined as the number of virus-infected cells among NIH3T3 cells (obtained from the American Type Culture Collection) on the occasion of allowing the virus-containing solution to coexist with those cells. For example, if 1 ml of a dilution (dilution factor 102 to 106) of the virus-containing solution is added to 3×10⁴ NIH3T3 cells occurring in each well (base area about 1.9 cm²) of a 24-well culture plate and the proportion of cells expressing the marker GFP is determined, the titer is given by the following calculation formula:

Viral vector titer=3×10⁴×dilution factor×proportion of GFP-expressing cells (cfu/ml).

Now, the infection of an early avian embryo with the replication-deficient retroviral vector so referred to herein is described.

An early embryo (preferably through a blood vein or the heart formed in the early embryo) formed after egg laying, except for the blastoderm stage immediately following egg laying, is infected with the replication-deficient retroviral vector. The means for the infection is not particularly restricted but includes the microinjection technique (Bosselman, R. A. et al. (1989): Science 243, 533), lipofection technique and eletroporation technique, among others; the microinjection technique is preferred, however.

The early embryo to be infected with the replication-deficient retroviral vector is desirably one obtained by at least 24 hours, after the start of incubation just after egg laying, of incubation of a fertilized avian egg in an incubation environment, for example in an environment of a temperature of 37.7 to 37.8° C. and a humidity of about 50 to 70% in the case of a chicken. More desirably, it is one after the lapse of at least 48 hours after the start of incubation. Preferably, it is an early embryo not later than 60 hours after the start of incubation. Such period of time varies depending on the external conditions such as incubation temperature and weather and, therefore, more precisely, an early embryo at the stage of 14 to 16 defined by Hamburger and Hamilton (H&H; Hamburger, V. and Hamilton, H. L. (1951), Morphol. 88, 49) is desirable. More desirable is an early embryo at the developmental stage of 15 according to the definition by H&H. Pulsation of the heart can be observed in an avian embryo in this developmental stage. The most desirable method of infection is the microinjection of the replication-deficient retroviral vector into the heart or a blood vessel connected thereto.

Thereafter, the incubation is continued and the avian early embryo infected with the replication-deficient retroviral vector is allowed to hatch. In the case of a chicken, for instance, a G0 transgenic chimera chick is born on day 21 after the start of incubation. The detailed procedure to be followed is as already disclosed by the present inventors (International Publication WO 2004/016081).

The G0 transgenic chimera bird constructed in accordance with the invention retains the foreign erythropoietin gene in all or part of somatic cells and/or germ cells thereof.

The G1 transgenic bird of the invention is produced by mating a G0 transgenic chimera bird holding a foreign erythropoietin gene in the germ cells thereof with a G0 transgenic chimera bird or a descendant thereof or a wild type bird. Thinkable mating types include the mating of a G0 transgenic male with a wild type female, of a G0 transgenic female with a wild type male, and of a G0 transgenic male with a G0 transgenic female, among others. It is also possible to backcross a descendant with a parent thereof. Among them, the type of mating of a G0 male with a wild type female is preferred from the efficiency viewpoint since one G0 male can be caused to mate with 3 to 10 wild type females.

The G0 male to be used for mating can be checked in advance for the presence or absence of the foreign erythropoietin gene in the sperm by the PCR technique, for instance. By using a G0 male in which the foreign erythropoietin gene has been confirmed, it becomes possible to obtain G1 transgenic birds with high efficiency.

The G1 transgenic bird constructed in accordance with the invention preferably retain the foreign erythropoietin gene uniformly in all somatic cells thereof. The distinction can be made by confirming the occurrence of the foreign erythropoietin gene in somatic cells such as blood cells by the PCR technique, for instance.

Transgenic birds of the G2 and succeeding generations of the invention can be produced by mating a G1 transgenic bird with a G1 transgenic bird or a descendant thereof or a wild type bird or a G0 transgenic chimera bird. Thinkable mating types include the mating of a G1 transgenic male with a wild type female, of a G1 transgenic female with a wild type male, and of a G1 transgenic male with a G1 transgenic female, among others. It is also possible to backcross a descendant with a parent thereof. Among them, the type of mating of a G1 male with a wild type female is preferred from the efficiency viewpoint since one G1 male can be caused to mate with 3 to 10 wild type females.

The method for producing erythropoietin according to the invention is characterized by recovering erythropoietin from the above-mentioned transgenic bird. More particularly, the method is characterized by recovering and purifying erythropoietin from the blood and/or ovum of the transgenic bird constructed. The methods of recovery and purification are not particularly restricted but includes, among others, chromatography techniques using various columns containing or utilizing an antibody, anion exchanger, reversed phase, gel filtration, hydroxyapatite, Blue Sepharose or phenylboric acid, for instance, ultrafiltration, precision filtration, centrifugation and/or combination of these.

### EFFECT OF THE INVENTION

The present invention provides a transgenic bird producing high-concentration erythropoietin and a method for constructing the same. The transgenic bird of the invention can produce erythropoietin showing a specific activity (specifically, specific activity with the proliferativity of erythropoietin-dependent cells being taken as an index) equal to or higher than that of the conventional erythropoietin species produced by CHO cells.

### BEST MODE FOR CARRYING OUT THE INVENTION

The following examples illustrate the present invention in detail. These examples are, however, by no means of the scope of the present invention.

### Example 1

### Formation of a Vector Construct for Expression of a Human Erythropoietin Gene

A vector construct pMSCV/GΔAhEPOW for expression of a human erythropoietin gene was prepared in the following manner.

1. A fragment containing a series of the murine phosphoglycerate kinase (PGK) promoter and the Neo^{r} gene was eliminated from pMSCVneo (product of Clontech Laboratories, Inc.) using the restriction enzymes BglII and BamHI, and the remaining vector fragment was allowed to self-ligate to construct the plasmid pMSCV.
2. A GFP gene fragment was excised from pGREEN LANTERN-1 (product of Gibco BRL) with the restriction enzyme NotI and inserted into pZeoSV2(+) (product of Invitrogen Corporation) at the NotI site. A plasmid having a structure resulting from insertion of the GFP gene in the same direction as the T7 promoter was named pZeo/GFP.
3. A GFP gene fragment was excised from pZeo/GFP with the restriction enzymes EcoRI and XhoI and joined to a vector fragment of pMSCV treated with the restriction enzymes EcoRI and XhoI to construct a plasmid, pMSCV/G.
4. A ΔAct promoter fragment was amplified from pMiwZ (Suemori et al., 1990, Cell Diff. Dev. 29:181-185) by PCR (94° C./15 seconds→50° C./30 seconds→68° C./1 minute: 10 cycles; 94° C./15 seconds→62° C./30 seconds→68° C./1 minute: 30 cycles; KOD-Plus-DNA polymerase (product of Toyobo Co., Ltd.)) using, as primers, two chemically synthesized oligonucleotides, 5′-acgcgtcgacgtgcatgcacgctcattg-3′ (SEQ ID NO:1, the underlined portion being the SalI restriction enzyme site) and 5′-acgcgtcgacaacgcagcgactcccg-3′ (SEQ ID NO:2, the underline portion being the SalI restriction enzyme site). The ΔAct promoter fragment was then excised using the restriction enzyme SalI and inserted into pETBlue-2 (product of Novagen) at the SalI site thereof, whereby a plasmid, pETBlue/ΔAct, was constructed.
5. The ΔAct promoter fragment was excised from pETBlue/ΔAct with the restriction enzyme SalI and inserted into pMSCV/G at the XhoI site thereof. A plasmid having a structure resulting from insertion of the ΔAct promoter in the same direction as the GFP gene was named pMSCV/GΔA.
6. Using two chemically synthesized oligonucleotide pairs, namely 5′-gcgccccaccacgcctcatctgtgacagcc-3′ (SEQ ID NO:3) and 5′-ggctgtcacagatgaggcgtggtggggcgc-3′ (SEQ ID NO:4), and 5′-gctctgggagcccagaaggaagccatctcc-3′ (SEQ ID NO:5) and 5′-ggagatggcttccttctgggctcccagagc-3′ (SEQ ID NO:6), together with QuickChange XL Site-Directed Mutagenesis Kit (product of Stratagene), two site-specific mutations were introduced into the human erythropoietin gene contained in the plasmid pcDNA3.1/GS/hEPO (Clone RG001720, product of Invitrogen) and thus a plasmid, pcDNA3.1/GS/αhEPO, containing the Amgen type human erythropoietin gene (GenBank Accession No. M11319) was constructed.
7. The human erythropoietin gene fragment (including the secretory signal sequence) was amplified from pcDNA3.1/GS/αhEPO by the PCR using two chemically synthesized oligonucleotides, 5′-agccaagcttaccatgggggtgcacgaa-3′ (SEQ ID NO:7) and 5′-cgataagcttacgcgttcatctgtcccctgtcctgcaggcctcc-3′ (SEQ ID NO:8) (each underlined sequence being a HindIII restriction enzyme site), as primers and, then excised using the restriction enzyme HindIII and inserted into pMSCV/GΔA at the HindIII site thereof. The plasmid having a structure resulting from insertion of the human erythropoietin gene in the same direction as that of the ΔAct promoter was designated as pMSCV/GΔAhEPO.
8. The WPRE sequence was amplified from pWHV8 (American Type Culture Collection 45097) by the PCR using two chemically synthesized oligonucleotides, namely 5′-ccatcgataatcaacctctggattacaaaatttgtga-3′ (SEQ ID NO: 9) and 5′-ccatcgatcaggcggggaggcg-3′ (SEQ ID NO:10) (each underlined sequence being a ClaI restriction site), as primers and inserted into pBluescript IIKS(−) (product of Stratagene) at the EcoRV site; a plasmid, pBlue/WPRE, was thus constructed. The pBlue/WPRE plasmid was prepared using the dam-Escherichia coli strain JM110 (product of Stratagene) as the host.
9. WPRE was excised from pBlue/WPRE using the restriction enzyme ClaI and inserted into pMSCV/GΔAhEPO at the ClaI site thereof. The plasmid having a structure resulting from insertion of WPRE with the SacII site in WPRE being remote from the human erythropoietin gene was designated as pMSCV/GΔAhEPOW.

The structure of the thus-constructed, replication-deficient retroviral vector-derived vector construct pMSCV/GΔAhEPOW is shown in FIG. 1. In FIG. 1, the ampicillin resistance gene Amp^{r}, virus packaging signal sequence ψ+ and long terminal repeat sequences 5′LTR and 3′LTR are all of pMSCVneo origin.

### Example 2

### Preparation of a Retroviral Vector for Expression of Human Erythropoietin Gene

For preparing a retroviral vector from the vector construct pMSCV/GΔAhEPOW constructed in Example 1, 5×106 packaging cells GP293 (product of Clontech Laboratories, Inc.) were sown and cultured in a culture dish having a diameter of 100 mm. The medium was replaced with fresh DMEM (Dulbecco's modified Eagle medium), and 8 μg of the pVSV-G vector (product of Clontech Laboratories, Inc.) and 8 μg of pMSCV/GΔAhEPOW were introduced into the above GP293 cells by the lipofection technique. After 48 hours, the culture supernatant containing virions was recovered and deprived of contaminants by passing through a 0.45-μm cellulose acetate filter (product of Advantech Co., Ltd.). Polybrene (product of Sigma) was added to the solution obtained to a concentration of 8 μg/ml and the resulting mixture was used as a virus-containing solution.

The virus-containing solution prepared was added to separately cultured GP293 cells and, after 48 hours of cultivation, the cells were cloned by the limiting dilution method. A stable packaging strain capable of strongly expressing GFP by virus infection was thus obtained.

The stable packaging strain obtained was cultured in a dish with a diameter of 100 mm until an 80% confluent state, and 16 μg of pVSV-G was introduced thereinto by the lipofection technique. After 48 hours, a culture supernatant containing virions was recovered.

This culture supernatant was centrifuged at 20,000×g and at 4° C. for 5 hours to settle the virions. The supernatant was removed, 20 μl of a solution containing 50 mM Tris-HCl (pH 7.8), 130 mM NaCl and 1 mM EDTA was added to the virion-containing precipitate and, after overnight standing at 4° C. and after thorough suspending, a virus-containing solution was recovered. The thus-obtained viral vector showed a titer of 2×10⁹ cfu/ml.

The viral vector titer measurement was carried out in the following manner. Thus, 1.5×10⁴ NIH/3T3 cells (American Type Culture Collection CRL-1658) were sowed in each well (base area about 1.9 cm²) of a 24-well culture plate. A 1-ml portion of each dilution (dilution factor: 102 to 106) of the virus-containing solution was added to 3×10⁴ cells expected to be attained after cultivation for all day and night in each well and, after 48 hours, the proportion of GFP-expressing cells was determined under a fluorescent microscope. The viral vector titer was calculated according to the formula given below.

Viral vector titer=3×10⁴×dilution factor×proportion of GFP-expressing cells (cfu/ml).

### Example 3

### Retroviral Vector Preparation in the Case of No Utilization of WPRE

In the same manner as in Example 2, a retroviral vector was prepared from the WPRE-free vector construct pMSCV/GΔAhEPO constructed in Example 1. The viral vector obtained had a titer of 4×10⁸ cfu/ml.

### Example 4

### Microinjection of a Retroviral Vector into Chicken Embryos and Artificial Hatching

Fertilized chicken eggs (Nippon Seibutsu Kagaku Kenkyusho) were placed on an incubator (Showa Furanki model P-008) with a built-in automatic egg rolling device in an environment maintained at 37.9° C. and a humidity of 65%, and this time was regarded as the time of starting incubation (hour 0). Incubation was carried out while turning the eggs at an angle of 90 degrees at 15-minute intervals.

After the lapse of about 55 hours from the start of incubation (at stage 15 according to the definition by H&H), the eggs were taken out of the incubator. A round portion 3.5 cm in diameter of the sharp end of each egg was cut off using a diamond cutter (MINOMO 7C710, product of Minitor Co., Ltd.) to expose the embryo on the yolk surface. Under a stereoscopic microscope, a glass capillary needle was stuck into the heart observed in this early embryo, followed by microinjection of about 2 μl of the virus-containing solution prepared in Example 2. The capillary needle used was prepared by processing a glass tube (GD-1, product of Narishige Co., Ltd.) using a micropipette producing device (PC-10, product of Narishige) and cutting off the tip of the needle obtained so that the outside diameter might amount to about 20 μm. For the microinjection, a microinjector (Transjector 5246, product of Eppendorf) was used.

An opening with a diameter of 4.5 cm was formed on the dull end of a double-yolked chicken egg prepared separately, and the contents were discarded. The contents of the fertilized egg after microinjection treatment were transferred to the remaining eggshell. A 0.5-ml portion of a 50 mg/ml calcium lactate suspension in egg white was added thereto and, then, the opening was closed with a poly(vinylidene chloride) wrap (Saran Wrap, product of Asahi Chemical Industry) using egg white as a paste. The whole was returned to the incubator. Incubation was carried out under oxygen feeding while turning the egg at an angle of 30 degrees at 1-hour intervals. On day 18 after the start of incubation, the egg turning was discontinued and, on day 21 when the chick began pecking, the egg shell was broken for hatching. The hatchability in this artificial hatching was 30 to 60%.

### Example 5

### Western Blot Analysis of the Serum of Each Chicken Born by Artificial Hatching and of the Egg Albumen and Egg Yolk

Each chick born as described in Example 4 was bred for growth. A blood sample was taken from the mature chicken through the basilic vein, the blood obtained was allowed to stand at room temperature for 30 minutes and then centrifuged at 15,000 rpm for 10 minutes, and the supernatant was used as a serum sample. This serum and the egg albumen and egg yolk of an egg laid by a female mature chicken were 20-fold diluted with PBS and subjected to SDS-PAGE. The gel concentration was 10%. A PVDF membrane blotted from this gel was immersed in a blocking solution (5% skimmed milk, TBS-0.05% Tween 20) and then reacted with a 2 μg/ml rabbit anti-human erythropoietin antibody (product of R&D Systems) as a primary antibody and then with a 1 μg/ml peroxidase-labeled goat anti-rabbit IgG antibody (product of Santa Cruz) as a secondary antibody. These reactions were carried out in the blocking solution at room temperature for 1 hour. Finally, the PVDF membrane and an ECL kit (product of Amersham) were used to expose an X-ray film, which was then developed (FIG. 2). As a result, the expression of human erythropoietin could be confirmed in all the serum samples derived from the four chickens subjected to analysis. In the eggs laid by two hens, the expression of human erythropoietin in the egg albumen was higher than in the serum samples, while the expression in the egg yolk was low. Thus, human erythropoietin-producing G0 transgenic chimera chickens could be produced.

### Example 6

### Determinations of Human Erythropoietin Expressed in Serum, Egg Albumen and Egg Yolk Samples Derived from Chickens Born by Artificial Hatching

Serum, egg albumen and egg yolk samples were prepared in the same manner as in Example 5 and assayed for human erythropoietin by the RIA technique using the Recombigen EPO kit (product of Mitsubishi Kagaku Iatron). As a result, the serum human erythropoietin concentration was above 2,000 I.U./ml in all the 6 chickens studied. An individual showed the highest concentration of 3,000 I.U./ml. While the average concentration in egg yolk was 1 I.U./ml, hence the expression was just about nil, 1,900 I.U./ml, on an average, of human erythropoietin was detected in the egg albumen, and an individual showed the highest concentration of 5,400 I.U./ml. FIG. 3 shows the changes in levels of expression in the egg albumen in the individual #111, indicating that the expression level of about 2,000 I.U./ml was maintained for one month. Thus, G0 transgenic chimera chickens producing human erythropoietin in high concentrations could be constructed.

### Example 7

### Measurement of the Activity of Human Erythropoietin Expressed in a Chicken Born by Artificial Hatching

The serum sample which showed a human erythropoietin concentration of 2,000 I.U./ml in Example 6 was subjected to activity measurement. The measurement was carried out by the cell proliferation assay using Baf/EpoR cells proliferating in an erythropoietin-dependent manner (Japanese Kokai Publication Hei-10-94393). The medium used for Baf/EpoR cells was RPMI 1640 liquid medium (product of Nissui Pharmaceutical) containing 5% fetal bovine serum, 50 units/ml penicillin and 50 μg/ml streptomycin.

Baf/EpoR cells were washed three times with the medium by repeating resuspension and centrifugation and then diluted to a concentration of 5.6×10⁴ cells/ml, and a 90-μl portion of the dilution was added to each well (base area about 0.3 cm²) of a 96-well culture plate. The above serum sample and a standard human erythropoietin sample (product of Calbiochem, derived from CHO, 2,000 I.U./ml) were each diluted, from 1,600 I.U./ml, with the medium in the manner of serial twofold dilution. A 10-μl portion of each dilution was added to each well of the 96-well culture plate sowed with the cells to give a uniform suspension. In a control experiment, a wild chicken-derived serum sample was used. Three wells were used for each dilution of each sample. After 2 days and nights of cultivation, 10 μl of the Cell Counting Kit-8 (product of Dojindo Laboratories) solution was added to each well. The color development reaction was allowed to proceed for about 1 to 4 hours and then terminated by adding 10 μl of 0.1 mol/ml hydrochloric acid to each well, and the absorbance (OD) of each well at 450 nm was measured using a microplate reader (product of BIO-RAD). For each dilution of each sample, the mean of the measurement results for the three wells was calculated. FIG. 4 shows the results of plotting of such mean values for each sample against the human erythropoietin concentration. The results revealed that the human erythropoietin in the serum sample had a specific activity (activity per unit mass) equivalent to or higher than that of the standard human erythropoietin. Thus, a G0 transgenic chimera chicken producing such active human erythropoietin could be constructed.

### BRIEF DESCRIPTION OF THE DRAWINGS

FIG. 1 This shows the structure of the vector construct pMSCV/GΔAhEPOW for expression of a human erythropoietin gene. Amp^{r} indicates the ampicillin resistance gene. PΔact indicates the β-actin promoter gene. ψ+ indicates the virus packaging signal sequence. hEPO indicates the human erythropoietin gene. 5′LTR and 3′LTR respectively indicate the long terminal repeat sequences of MSCV.

FIG. 2 This shows the results of western blot analysis of the serum, egg albumen and egg yolk samples derived from chickens born by artificial hatching following microinjection, into chicken embryos, of the retroviral vector for expression of a human erythropoietin gene. # indicates the individual number, and NT indicates a wild chicken. Each arrow indicates the site corresponding to the molecular weight of erythropoietin.

FIG. 3 This shows the changes with time of the human erythropoietin in the albumen of an egg laid by a chicken born by artificial hatching following microinjection, into a chicken embryo, of the retroviral vector for expression of a human erythropoietin gene.

FIG. 4 This shows the results of the activity measurement, using erythropoietin-dependent cells, of the human erythropoietin in the serum of a chicken born by artificial hatching following microinjection, into a chicken embryo, of the retroviral vector for expression of a human erythropoietin gene. The abscissa denotes the human erythropoietin concentration, and the ordinate denotes the result of measurement using Cell Counting Kit-8. The term “standard” indicates standard human erythropoietin, G0 (#103) indicates a G0 transgenic chimera chicken-derived serum sample, and Wild indicates a wild chicken-derived serum sample.

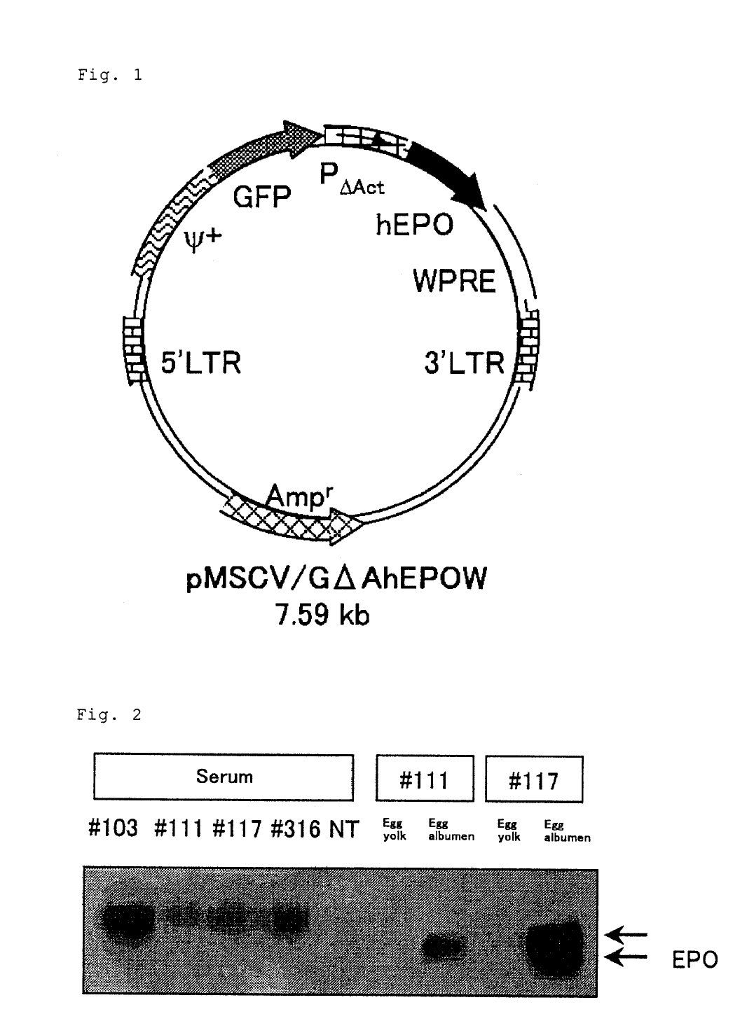

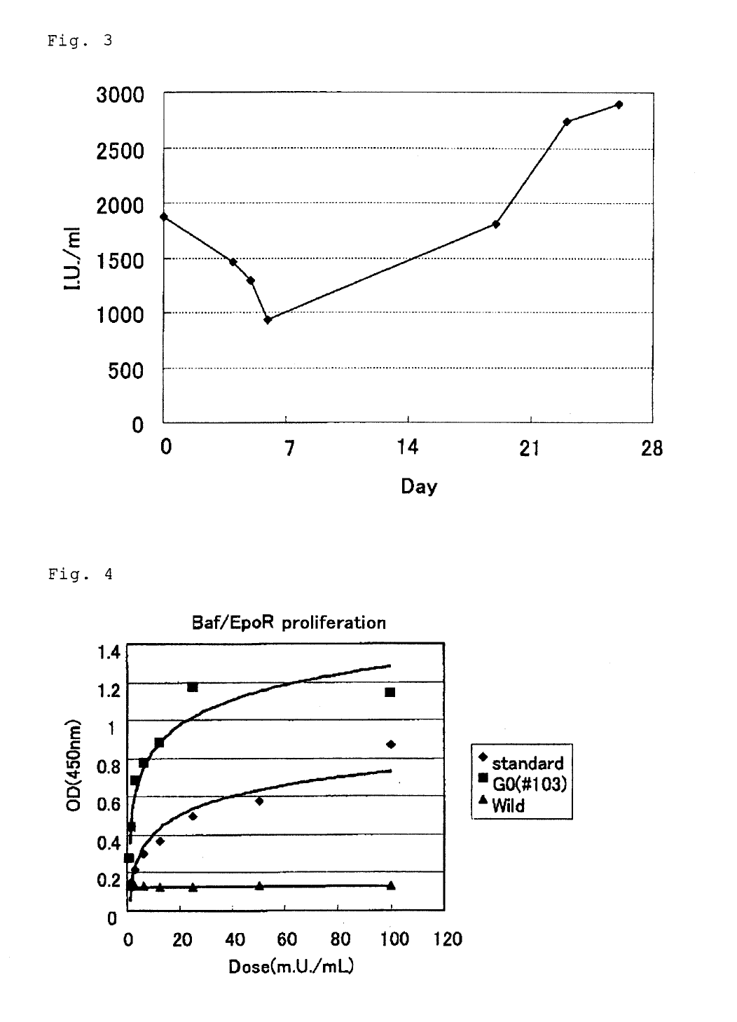

1. A G0 transgenic chimera bird as obtained by incubating a fertilized avian egg, infecting the early embryo formed after egg laying, except for the blastoderm stage immediately following egg laying, with a replication-deficient retroviral vector containing a foreign erythropoietin gene and allowing the embryo to hatch.

2. The G0 transgenic chimera bird according to claim 1,
wherein the foreign erythropoietin gene is a mammal-derived one.

3. The G0 transgenic chimera bird according to claim 2,
wherein the foreign erythropoietin gene is a human-derived one.

4. The G0 transgenic chimera bird according to any one of claim 1,
wherein the foreign erythropoietin gene is under the control of a non-tissue-specific promoter.

5. The G0 transgenic chimera bird according to claim 4,
wherein the non-tissue-specific promoter is a chicken β-actin gene-derived one.

6. The G0 transgenic chimera bird according to any one of claim 1,
wherein the foreign erythropoietin gene is under the control of a tissue-specific promoter.

7. The G0 transgenic chimera bird according to claim 6,
wherein the tissue-specific promoter is a chicken ovalbumin gene-derived one.

8. The G0 transgenic chimera bird according to any one of claim 1,
wherein the foreign erythropoietin gene has a posttranscriptional regulatory element added thereto.

9. The G0 transgenic chimera bird according to claim 8,
wherein the posttranscriptional regulatory element is WPRE.

10. The G0 transgenic chimera bird according to any one of claim 1,
wherein the replication-deficient retroviral vector is a MoMLV- or MSCV-derived one.

11. The G0 transgenic chimera bird according to any one of claim 1,
wherein the replication-deficient retroviral vector is of the VSV-G pseudotype.

12. The G0 transgenic chimera bird according to any one of claim 1,
wherein the infection is carried out using, as the replication-deficient retroviral vector, a virus-containing solution having a replication-deficient retroviral vector titer of not lower than 1×10⁸ cfu/ml but not higher than 1×10¹⁰ cfu/ml.

13. The G0 transgenic chimera bird according to any one of claim 1,
wherein the early embryo is one formed not earlier by 24 hours but not later by 60 hours than the start of incubation.

14. The G0 transgenic chimera bird according to claim 13,
wherein the early embryo is one formed not earlier by 48 hours but not later by 60 hours than the start of incubation.

15. The G0 transgenic chimera bird according to any one of claim 1,
wherein the infection is carried out by microinjection into the heart or a blood vessel formed in the early embryo.

16. The G0 transgenic chimera bird according to claim 15,
wherein the infection is carried out by microinjection into the heart formed in the early embryo.

17. A G1 transgenic bird and a descendant thereof as obtained by mating the G0 transgenic chimera bird according to any one of claim 1 with another G0 transgenic chimera bird of the same kind, a descendant thereof or a wild type bird.

18. A G2 transgenic bird and a descendant thereof as obtained by mating the G1 transgenic bird according to any of claim 17 with another G1 transgenic bird of the same kind, a descendant thereof, a wild type bird or a G0 transgenic chimera bird obtained by incubating a fertilized avian egg, infecting embryo formed after egg laying, except for the blastoderm stage immediately following egg laying, with a replication-deficient retroviral vector containing a foreign erythropoietin gene and allowing the embryo to hatch.

19. The transgenic bird according to claim 1
which is a chicken or quail.

20. An egg laid by the transgenic bird according to any one of claim 1.

21. A method for producing erythropoietin
which comprises extracting and purifying erythropoietin from the egg according to claim 20.

22. A method for producing erythropoietin
which comprises extracting and purifying erythropoietin from the blood of the transgenic bird according to claim 1.

23. A sperm of the transgenic bird according to claim 1.

24. A method for constructing a G0 transgenic chimera bird
which comprises incubating a fertilized avian egg, infecting the early embryo formed after egg laying, except for the blastoderm stage immediately following egg laying, with a replication-deficient retroviral vector containing a foreign erythropoietin gene and allowing the embryo to hatch.

25. The method for constructing a G0 transgenic chimera bird according to claim 24,
wherein the foreign erythropoietin gene is a mammal-derived one.

26. The method for constructing a G0 transgenic chimera bird according to claim 25,
wherein the foreign erythropoietin gene is a human-derived one.

27. The method for constructing a G0 transgenic chimera bird according to claim 24,
wherein the foreign erythropoietin gene is under the control of a non-tissue-specific promoter.

28. The method for constructing a G0 transgenic chimera bird according to claim 27,
wherein the non-tissue-specific promoter is a chicken β-actin gene-derived one.

29. The method for constructing a G0 transgenic chimera bird according to claim 24,
wherein the foreign erythropoietin gene is under the control of a tissue-specific promoter.

30. The method for constructing a G0 transgenic chimera bird according to claim 29,
wherein the tissue-specific promoter is a chicken ovalbumin gene-derived one.

31. The method for constructing a G0 transgenic chimera bird according to claim 24,
wherein the foreign erythropoietin gene has a posttranscriptional regulatory element added thereto.

32. The method for constructing a G0 transgenic chimera bird according to claim 31,
wherein the posttranscriptional regulatory element is WPRE.

33. The method for constructing a G0 transgenic chimera bird according to claim 24,
wherein the replication-deficient retroviral vector is a MoMLV- or MSCV-derived one.

34. The method for constructing a G0 transgenic chimera bird according to claim 24,
wherein the replication-deficient retroviral vector is of the VSV-G pseudotype.

35. The method for constructing a G0 transgenic chimera bird according to claim 24,
wherein the infection is carried out using, as the replication-deficient retroviral vector, a virus-containing solution having a replication-deficient retroviral vector titer of not lower than 1×10⁸ cf/ml but not higher than 1×10¹⁰ cfu/ml.

36. The method for constructing a G0 transgenic chimera bird according to claim 24,
wherein the early embryo is one formed not earlier by 24 hours but not later by 60 hours than the start of incubation.

37. The method for constructing a G0 transgenic chimera bird according to claim 36,
wherein the early embryo is one formed not earlier by 48 hours but not later by 60 hours than the start of incubation.

38. The method for constructing a G0 transgenic chimera bird according to claim 24,
wherein the infection is carried out by microinjection into the heart or a blood vessel formed in the early embryo.

39. The method for constructing a G0 transgenic chimera bird according to claim 38,
wherein the infection is carried out by microinjection into the heart formed in the early embryo.


SEQUENCE LISTING (10 sequences)

SEQ ID NO: 1 - DNA, 28 bp, Artificial
  Sequence of a designed oligonucleotide acting as the 5′-primer for PCR amplification of the chicken b-actin promoter fragment lacking the intron with the Sal I recognition site at the 5′-terminal
acgcgtcgacgtgcatgcacgctcattg

SEQ ID NO: 2 - DNA, 26 bp, Artificial
  Sequence of a designed oligonucleotide acting as the 3′-primer for PCR amplification of the chicken b-actin promoter fragment lacking the intron with the Sal I recognition site at the 5′-terminal
acgcgtcgacaacgcagcgactcccg

SEQ ID NO: 3 - DNA, 30 bp, Artificial
  Sequence of a designed oligonucleotide acting as a primer for PCR amplification with site-directed mutagenesis for the 94th nucleotide from the ATG of a human erythropoietin cDNA
gcgccccaccacgcctcatctgtgacagcc

SEQ ID NO: 4 - DNA, 30 bp, Artificial
  Sequence of a designed oligonucleotide acting as a primer for PCR amplification with site-directed mutagenesis for the 94th nucleotide from the ATG of a human erythropoietin cDNA
ggctgtcacagatgaggcgtggtggggcgc

SEQ ID NO: 5 - DNA, 30 bp, Artificial
  Sequence of a designed oligonucleotide acting as a primer for PCR amplification with site-directed mutagenesis for the 427th nucleotide from the ATG of a human erythropoietin cDNA
gctctgggagcccagaaggaagccatctcc

SEQ ID NO: 6 - DNA, 30 bp, Artificial
  Sequence of a designed oligonucleotide acting as a primer for PCR amplification with site-directed mutagenesis for the 427th nucleotide from the ATG of a human erythropoietin cDNA
ggagatggcttccttctgggctcccagagc

SEQ ID NO: 7 - DNA, 28 bp, Artificial
  Sequence of a designed oligonucleotide acting as the 5′-primer for PCR amplification of a human erythropoietin cDNA with the Hind III recognition site at the 5′-terminal
agccaagcttaccatgggggtgcacgaa

SEQ ID NO: 8 - DNA, 44 bp, Artificial
  Sequence of a designed oligonucleotide acting as the 3′-primer for PCR amplification of a human erythropoietin cDNA with the Hind III recognition site at the 5′-terminal
cgataagcttacgcgttcatctgtcccctgtcctgcaggcctcc

SEQ ID NO: 9 - DNA, 37 bp, Artificial
  Sequence of a designed oligonucleotide acting as the 5′-primer for PCR amplification of WRPE with the Cla I recognition site at the 5′-terminal
ccatcgataatcaacctctggattacaaaatttgtga

SEQ ID NO: 10 - DNA, 22 bp, Artificial
  Sequence of a designed oligonucleotide acting as the 3′-primer for PCR amplification of WRPE with the Cla I recognition site at the 5′-terminal
ccatcgatcaggcggggaggcg